(19) United States
(12) Patent Application Publication
(10) Pub. No.: US 2006/0116631 A1
(43) Pub. Date: Jun. 1, 2006

(54) Needleless injector

(76) Inventor: Jim Fukushima, Orange, CA (US)

Correspondence Address: CHRISTIE, PARKER & HALE, LLP, PO BOX 7068, PASADENA, CA, 91109-7068, US

(21) Appl. No.: 11/158,488

(22) Filed: Jun. 22, 2005

Related U.S. Application Data
(60) Provisional application No. 60/582,859, filed on Jun. 24, 2004.

(51) Int. Cl. A61M 5/30

(52) U.S. Cl. 604/68

(57) ABSTRACT

Needleless injectors for injecting fluids subcutaneously without a needle are generally discussed herein with particular discussions on needleless injectors for injecting a volume of fluid through two or more nozzles. The injectors can have a spring driven force generating component or a gas driven type. The injectors can have a fluid handling component having a single integrally molded component or one that is multi-component. The entire injector can be disposable or, alternatively, only the fluid handling component is disposable. The force generating component can be reset for use with a new or scone fluid handling component. Finally, the two or more discharge nozzles can be located at the discharge end of a single ampule. Alternatively, the two or more nozzles can be multiple single orifice ampules mounted on one or more force generating components.

Needleless injectors for injecting fluids subcutaneously without a needle are generally discussed herein with particular discussions on needleless injectors comprising multiple orifices.

### CROSS-REFERENCE TO RELATED APPLICATION

This is a ordinary application of Ser. No. 60/582,859, filed Jun. 24, 2004, the contents of which are expressly incorporated herein by reference as if set forth in full.

### BACKGROUND

Jet injection devices are well known in the art for administering intramuscular and subcutaneous medications without needles. Exemplary hypodermic jet injectors are described in U.S. Pat. Nos. 5,499,972; 5,569,189; 5,704,911, and 6,558,348, their contents are hereby expressly incorporated herein by reference.

In general, these patents disclose a hypodermic jet injector assembly having an ampule for holding liquid medicament or medication and a jet injector or force generating component for injecting medication contained within the ampule subcutaneously without a needle. The ampule is generally a single integral component made from a thermoplastic material having a nozzle on one end for discharging medication, the discharge end, and an opening on the other end for securing the ampule to a jet injector, the inlet end. The ampule's physical characteristics, such as wall thickness and diameter, are determined in part by the dosage capacity, the plunger type, the nozzle size, and the operating pressure for delivering fluid subcutaneously without a needle.

The force generating component includes a metallic cylinder or a thermoplastic housing enclosed on one end and open on the other end for receiving an ampule. Within the cylinder, the components of the jet injector generally include a spring, a piston, a shaft, a plunger, and a trigger. The jet injector device operates by cocking or compressing the spring, which is in mechanical communication with the piston. The trigger is used to release the spring, which drives the piston, which then drives the shaft, and which then drives the plunger into the medication to discharge the medication out the nozzle. A typical operating pressure for a jet injector device is in the range of about 3,000 to 3,500 psi at the nozzle, with a much higher pressure range of about 5,000 to 6,000 psi developed during the initial thrust of the piston, although this range may vary depending on some of the variables previously discussed. Thus, a suitable ampule for use with the jet injector is one that is capable of handling the aforementioned pressure range.

Subsequent to discharging the injector, the ampule, plunger, and shaft may be separated from the force generating component and discarded. The force generating component, however, can be re-used by resetting the spring, as disclosed in the '911 patent. A new ampule, plunger, and shaft may then be connected to the force generating component by threading the ampule into the receiving end of the cylinder of the force generating component.

Another hypodermic jet injector example is disclosed in U.S. Pat. No. 6,558,348, entitled “Low Cost Disposable Needleless Injector System for Variable and Fixed Dose Applications”, the contents of which are expressly incorporated herein by reference. The '348 patent discloses a jet injector assembly designed for low cost production and for disposability after a single use. The disposable jet injector assembly generally comprises an ampule threadedly or permanently attached to a jet injector. The components within the jet injector typically include a spring in dynamic communication with a shaft and a piston.

The ampule may be threadedly or permanently attached to the jet injector. The shaft, via the extension, allows medication to be drawn into the ampule when it is retracted from a first position to a second position, which creates a vacuum in the ampule to thereby draw in medication. The injector assembly is used by placing the discharge nozzle next to the skin and then firing the trigger, as discussed above with reference to the re-useable jet injector model.

Although both the disposable and the re-useable jet injector assemblies are effective, reliable, and economical, there remains a need for alternative needleless injectors.

### SUMMARY

Embodiments of the present invention comprise an ampule and a force generating mechanism for delivering medicament located inside the ampule subcutaneously without a needle.

Preferably, the needleless injector provided in accordance with aspects of the present invention comprises an ampule for use with a force generating component to discharge fluid subcutaneously comprising a discharge end, a connection end, and a housing body defining an interior cavity disposed therebetween, a plunger slidably movable within the interior cavity of the housing body, and wherein the discharge end comprises a plurality of nozzles passing through at least one raised surface.

In another aspect of the present invention, there is provided an ampule for use with a force generating component to discharge fluid subcutaneously comprising a discharge end, a connection end, and a housing body defining an interior cavity disposed therebetween, and wherein the discharge end comprises two or more nozzles positioned at an angle to one another.

In still yet another aspect of the present invention, there is provided an ampule for use with a force generating component to discharge fluid subcutaneously comprising a discharge end comprising multiple discharge means for discharging fluid, a connection means, a housing body defining an interior cavity disposed therebetween, and a plunger means for moving fluid through the multiple discharge means to simultaneously discharge the fluid through the multiple discharge means.

Other aspects of the invention include a reusable spring injector, a disposable spring injector, and a gas driven spring injector.

Two or more than two nozzles may be incorporated for delivering fluid through the two or more nozzles subcutaneously without a needle.

The nozzles may be aligned parallel to one another or angled so that they either converge or diverge.

Other aspects and features of the present invention will become clearer when the specification, drawings, and claims are referred to as hereinafter described.

### BRIEF DESCRIPTION OF THE DRAWINGS

These and other features and advantages of the present invention will become appreciated as the same become better understood with reference to the specification, claims and appended drawings wherein:

FIG. 1 is a semi-schematic partial transparent side view of an injector comprising a fluid handling component connected to a force generating component provided in accordance with aspects of the present invention;

FIG. 2 is a semi-schematic partial transparent view of the fluid handling component of FIG. 1;

FIGS. 3-3C are semi-schematic views of the housing of the fluid handling component of FIG. 2;

FIG. 4 is a discharge end plate of the housing of FIGS. 3-3C;

FIG. 5 is a semi-schematic cross-sectional side view of the discharge end plate of FIG. 4 taken along line F5-F5;

FIG. 6 is a semi-schematic partial cross-sectional side view of the housing body of FIGS. 3-3C;

FIG. 7 is a semi-schematic partial cross-sectional side view of an alternative housing body provided in accordance with aspects of the present invention;

FIG. 8 is a semi-schematic partial cross-sectional side view of an alternative discharge end plate provided in accordance with aspects of the present invention;

FIG. 9 includes various views of a plunger assembly provided in accordance with aspects of the present invention;

FIG. 10 is a semi-schematic partial cross-sectional side view of the plunger of FIG. 9, shown with an O-ring;

FIG. 11 is a semi-schematic cross-sectional side view of an injector assembly comprising a reusable force generating component and a disposable ampule provided in accordance with aspects of the present invention; and

FIG. 12 is a semi-schematic cross-sectional side view of an injector assembly comprising a disposable force generating component and a disposable ampule provided in accordance with aspects of the present invention.

### DETAILED DESCRIPTION

The detailed description set forth below in connection with the appended drawings is intended as a description of the presently preferred embodiments of needleless injectors provided in accordance with aspects of the present invention and is not intended to represent the only forms in which the present invention may be constructed or utilized. The description sets forth the features and the steps for constructing and using embodiments of the jet injectors of the present invention in connection with the illustrated figures. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features.

Referring now to FIG. 1, a semi-schematic partial transparent side view of a needleless injector (herein “injector”) provided in accordance with aspects of the present invention is shown, which is generally designated 10. The injector 10 comprises a force generating component 12 and a fluid handling component 14. In one exemplary embodiment, the force generating component 12 may be any one of the force generating components disclosed in U.S. Pat. Nos. 5,499,972; 5,569,189; 5,704,911, and 6,558,348, which have previously been incorporated by reference.

Generally speaking, the force generating component 12 comprises a housing 16, a piston 18 movable by a spring mechanism 20, and a trigger for releasing the spring 20 to propel the piston. The fluid handling component 14 comprises a housing 24 defining an interior cavity having a connection end 26, a discharge end 28, and a plunger 30 slidably received therein. In one exemplary embodiment, the connection end 26 comprises male threads for treaded engagement with corresponding female threads on the force generating component 12. However, a reverse tread configuration may be incorporated without deviating from the spirit and scope of the present invention. As used herein, the injector proximal end 32 is the end closest to an operator of the jet injector 10 while the injector distal end 34 is the end furthest from the user, or closest to the subject to be injected if the two or the same person.

The piston 18 is configured to abut and move the plunger 30 distally when the injector 10 is activated. Thus, when the injector 10 is placed against the skin of a subject and injected by depressing the trigger 22, the spring 20 expands and pushes the piston 18 distally which then pushes the plunger 30 distally. As the plunger 30 moves distally, it moves the fluid stored inside the interior cavity of the housing 24 distally out of the discharge end 28, and, with sufficient force, the fluid pierces the skin and penetrates the subject subcutaneously without a needle. A typical operating pressure for a jet injector device to pierce a subject's skin and penetrate subcutaneously without a needle is in the range of about 3,000 to 3,500 psi at the nozzle, with a much higher pressure range of about 5,000 to 6,000 psi developed during the initial thrust of the piston.

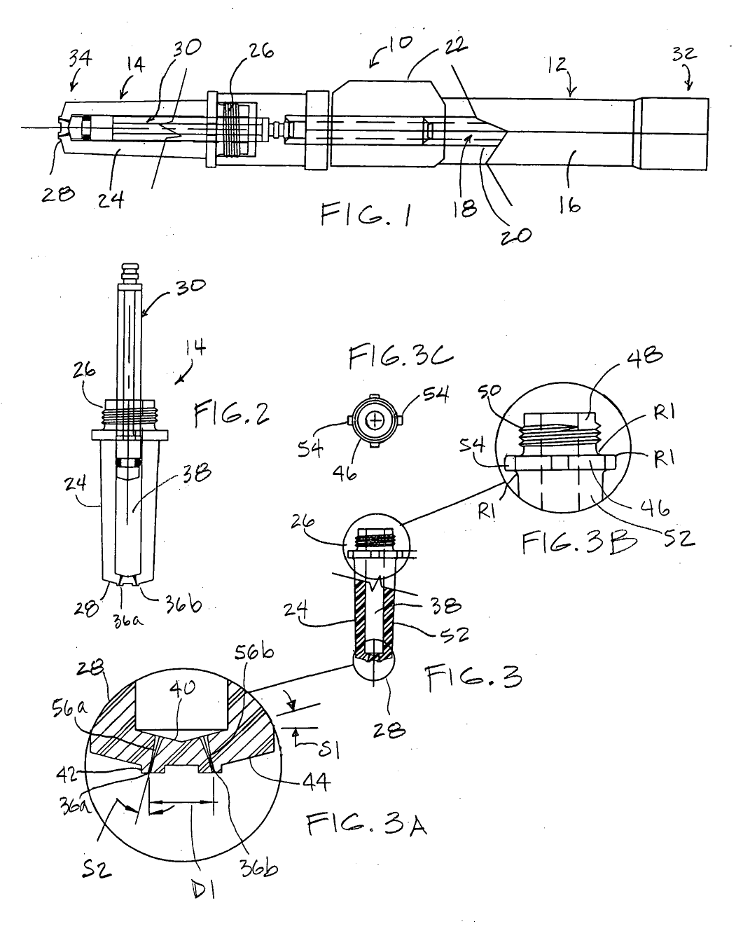

Referring now to FIG. 2, a semi-schematic transparent view of the fluid handling component 14 provided in accordance with aspects of the present invention is shown. In one exemplary embodiment, the fluid handling component 14 is an ampule and comprises a multi-orifice discharge end 28. Preferably, two orifices 36a, 36b and two flow nozzles 56a, 56b (FIG. 3A) are incorporated at the discharge end 28 for delivering fluid contained within the interior cavity 38 of the housing 24 subcutaneously without a needle. Alternatively, three or more orifices and corresponding number of nozzles may be incorporated without deviating from the spirit and scope of the present invention.

Broadly speaking, the two orifices at the discharge end 28 divides a given dosage into two smaller volume segments or smaller quantities for delivering the total dosage to a subject over two regions subcutaneously and concurrently. The dosage is, in effect, delivered over a larger area (e.g., over two areas as oppose to a single area under a single orifice). Thus, for a given dosage, a smaller volume is delivered through each orifice of a multi-orifice ampule as compared to the same dosage delivered by a single orifice ampule. All things being equal, lower volume through a given orifice means a smaller volume the subject will have to accommodate subcutaneously under that orifice, which, theoretically, means less pain and a more rapid injection. The same is true for a three or more orifice ampule.

In one exemplary embodiment, the operating pressure range for the multi-orifice ampule 14 is about 2,700 psi to about 3,700 psi at the discharge end. However, as previously discussed, orifice size, wall thickness, and plunger type are variables that may affect the operating pressure range. Another variable is the skin tissues of the subject to be injected. Tests have shown that higher operating pressure is required to penetrate a subject with darker skin than for a subject with lighter skin color.

FIGS. 3-3C depict various views of an exemplary ampule housing 24 provided in accordance with aspects of the present invention. In one exemplary embodiment, the housing 24 is made from a thermoplastic material or a plastic blend with exemplary thermoplastic materials including polycarbonate (PC), polyethylene terephthalate (PET), ethylene vinyl alcohol (EVOH), polyvinylidene chloride (PVDC), or their equivalents, with PC being more preferred. The housing dimensions may vary and are dependent on the volume capacity and operating pressure of the injector 10. For a 1 cc ampule using polycarbonate, the housing outside diameter of about 0.614″ and inside diameter of about 0.25″ are considered sufficiently adequate.

FIG. 3A is an enlarged view of the discharge end 28 of the ampule. In one exemplary embodiment, the interior distal end surface 40 of the discharge end 28 is cone shaped and comprises a sloped surface S1 of about 15 degrees. In one exemplary embodiment, a raised ring 42 is incorporated on the exterior distal end 44 of the housing, which has the two orifices 36a, 36b formed thereon. Two nozzles 56a, 56b are incorporated at the distal end, which terminate as nozzles 36a, 36b at the exterior surface of the ring 42. The two nozzles 56a, 56b are preferably angled so that as fluid flows through the nozzles and exits the orifices 36a, 36b, the two streams diverge as they exit the discharge end 28. This allows the injected fluid to be dispersed over a larger area subcutaneously. In a prefer embodiment, the nozzles 56a, 56b, with the orifices 36a, 36b forming the ends thereof, are angled S2 about plus 15 degrees and about minus 15 degrees from vertical, respectively. Alternatively, the two nozzles may align vertically and parallel to one another or are formed at angles other than 15 degrees from vertical. Still alternatively, the nozzles may be angled such that the streams exiting the orifices will converge instead of diverge, which is less preferred. For a 1 cc ampule, the gap D1 between the two nozzles 36a, 36b is about 0.173″ (FIG. 3A). Alternatively, the raised ring 42 may be excluded and the orifices 36a, 36b formed flushed with the exterior wall surface. Still alternatively, a plurality of raised protrusions equaling the number of nozzles may be incorporated instead of the continuously formed ring.

FIG. 3B is an end view of the connection end 26 of the ampule housing 24. The connection end 26 comprises a threaded stem 48 comprising a plurality of threads 50. A flange 46 is disposed between the threaded stem 48 and the housing body 52. In one exemplary embodiment, the various corners R1 are incorporated and are rounded with a radius of about 0.3″ to about 1.0″. A plurality of teeth 54 are optionally formed on the periphery of the flange 46. In one exemplary embodiment, four teeth 54 are incorporated. The teeth 54 extend the reach or size of the flange 46 without enlarging the flange, per se. However, the flange 46 may be extended radially outwardly (i.e., larger diameter) without deviating from the spirit and scope of the present invention.

FIG. 3C is an end view of the housing 24 looking from the perspective of the connection end 26 towards the discharge end 28. From the view shown, the teeth 54 can be seen disposed equally spaced apart along the periphery of the flange 46. Although four teeth 54 are shown, two, three, or more than four may be incorporated without deviating from the spirit and scope of the present invention.

FIG. 4 is an end view of the discharge end 28 or discharge end plate 55, which shows the following components: the exterior distal end surface 44, the raised protrusion or ring 42, and the two orifices 36a, 36b. For discussion purposes, the discharge end plate 55 has been identified as quadrants Q1-Q4. FIG. 5 is a cross-sectional side view of the discharge end plate 55 taken along line F5-F5 of FIG. 4, which depicts quadrants Q1/Q2. As shown, the two nozzles 56a, 56b each comprises an entry section 58 and an exit section 60, closest to the orifice 36. In one exemplary embodiment, the exit section 60 is about 0.040″ long and about 0.007″ in diameter. In one exemplary embodiment, the entry section 58 makes up the balance of the length of the nozzle and comprises a draft angle of about 15 degrees.

In one exemplary embodiment, the end plate 55 is separately formed from the housing body 52. The housing body 52 and the end plate 55 are thereafter heat welded together to form the housing 24. In one exemplary embodiment, laser welding is used to weld the end plate to the housing body 52. An exemplary laser includes a diode laser. However, other prior art welding methods for joining a first thermoplastic component to a second thermoplastic component may be used without deviating from the spirit and scope of the present invention. Alternatively, the end plate 55 may be co-molded to the housing body 52. Still alternatively, the end plate 55 may be made from a first material and the housing body 52 from a second material. The two materials are preferably thermoplastic materials.

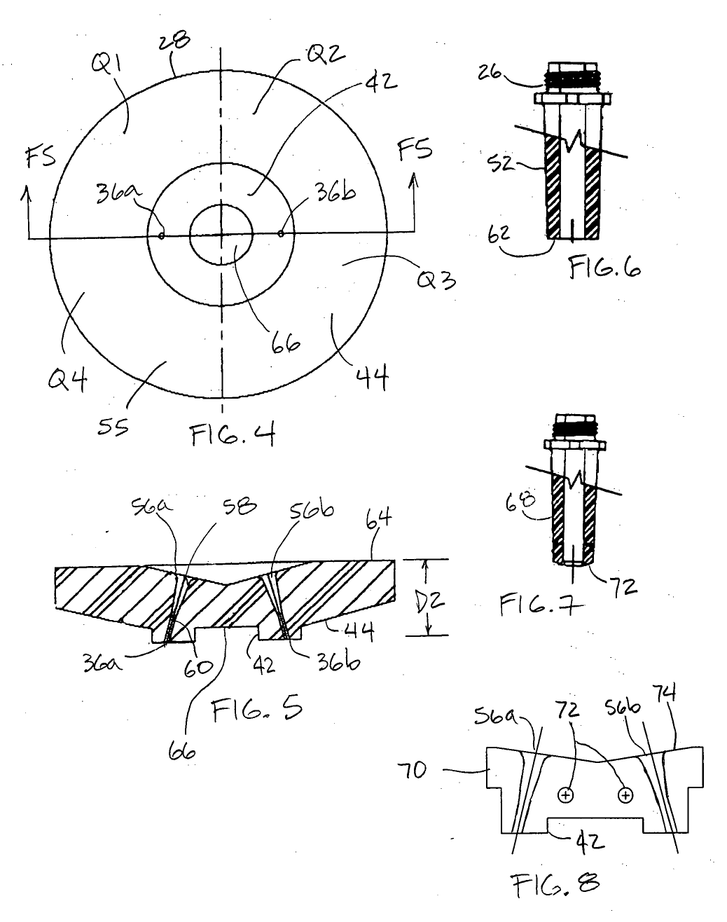

Referring now to FIG. 6 in combination with FIG. 5, in one exemplary embodiment, the connection end 26 is integrally formed with the housing body 52. A generally flat distal end edge 62 is incorporated to mate with a corresponding seat 64 on the discharge end plate 55. The seam defined by the end edge 62 of the housing body 52 and the seat 64 of the end plate 55 is welded, as previously discussed, to form the housing 24. In one exemplary embodiment, the distance D2 measured from the seat 64 to the tip of the raised ring 42 is about 0.120″, with the raised ring 42 measured from the end face 66 being about 0.021″.

In one exemplary embodiment, the end plate 55 comprises two or more separately formed and subsequently joined components having two, three, or more nozzles. In one particular embodiment, the end plate 55 is formed from two plate sections Q1/Q2 and Q3/Q4, which divide the end plate 55 in two with the seam intersecting the two nozzles 56a, 56b. The two sections Q1/Q2 and Q3/Q4 are preferably joined using known prior art welding methods, e.g., laser welding using, for example, diode laser.

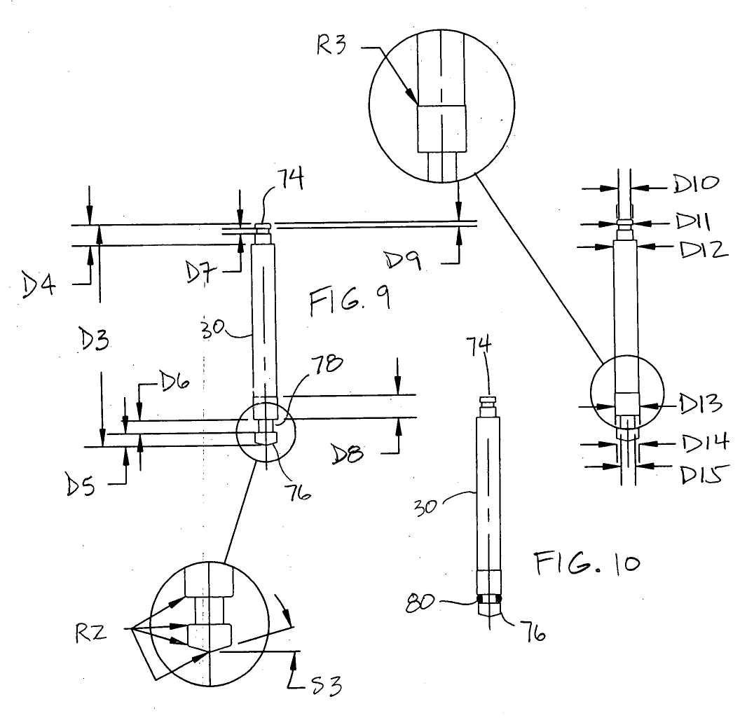

FIGS. 7 and 8 show an alternative housing body 68 and end plate 70. In the present alternative embodiment, the end edge 72 of the valve body 68 is tapered to form a taper abutment with a tapered seat 74 on the end plate 70. A pair of alignment dimples or bosses 72 are formed on one plate section Q1/Q2 to mate with a pair of alignment pins (not shown) incorporated on the corresponding plate section (e.g., Q3/Q4). The alignment dimples and pins facilitate alignment of the two plate sections Q1/Q2 and Q3/Q4 for joining purposes.

Although the two plate sections are described as being Q1/Q2 and Q3/Q4, they may be formed as Q1/Q4 and Q2/Q3, or any other combinations of quadrants. Still alternatively, while the nozzles 56a, 56b are described as diverging along the Q1/Q2 and Q3/Q4 seam, they may diverge by angling the nozzles to direct fluid flow out of the nozzles, for one nozzle, along the Q4 to Q1 quadrants, and for the other nozzle, along the Q2 to Q3 quadrants. In yet other aspects of the present invention, the nozzles may be angled in other than the angles described. In yet another exemplary embodiment, the nozzles are vertical and parallel to one another, which allows the end plate to be singularly formed and subsequently attached to the housing body or integrally formed with the housing body.

As previously discussed, by incorporating multiple orifices on the discharge end of a fluid handling component, smaller dosages may be delivered through each orifice as compared to the same total dosage delivered through a single orifice ampule. This configuration allows the multi-orifice fluid handling component to be used in higher dosage applications, such as a 1 ml dosage application without delivering a large bolus to a single injection site. Another exemplary application for the multi-orifice fluid handling component is for veterinary use, such as for example dogs and cats.

FIG. 9 depicts an exemplary plunger 30 provided in accordance with aspects of the present invention. The plunger 30 comprises a contact end 74, for contacting the piston 18 of the force generating component 12, and a push end 76, for pushing fluid out the discharge nozzles 56a, 56b. In one exemplary embodiment, the plunger 30 is made from a thermoplastic material. More preferably, the plunger is made from a polycarbonate material. Alternatively, other thermoplastics or plastic blends may be used, such as polycarbonate having approximately 15% glass or a glass loaded polymer of equivalent performance such as A model (a federally registered trademark of Amoco Oil Company of Chicago, Ill.). The dimensions of a plunger usable with a 1 cc ampule may be as follows:

| | | | |
|---|---|---|---|
| D3 = 2.131″ | D4 = 0.215″ | D5 = 0.1285″ | D6 = 0.131″ |
| D7 = 1/16″ | D8 = 0.220″ | D9 = 1/16″ | D10 = 0.131″ |
| D11 = 0.160″ | D12 = 0.232″ | D13 = 0.246″ | D14 = 0.241″ |
| D15 = 0.146″ | R2 = 0.2″ radius | S3 = 15 degrees | R3 = 0.1″ radius |

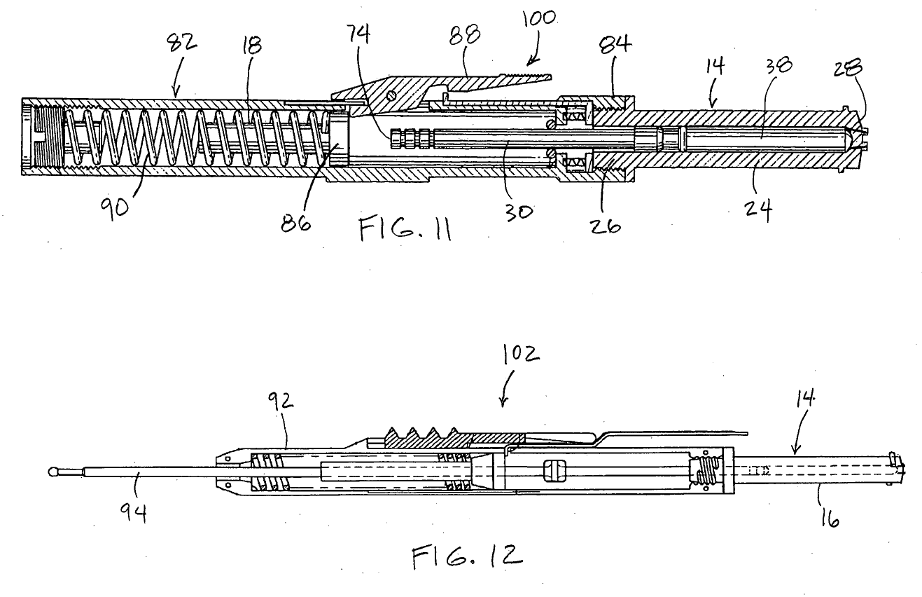

FIG. 10 shows an O-ring 80 mounted in a groove 78 located near the push end 76 of the plunger 30. In one exemplary embodiment, the O-ring is made from a silicone material with Teflon material being optional. The O-ring should be sized to have a slight compression when mounted inside the interior cavity 38 of the of the ampule housing 24. In one exemplary embodiment, the O-ring has a 1-4 thousands total crush or compression when mounted on the plunger 30 and placed inside the interior cavity of the housing. Alternatively, two or more grooves may be incorporated for accommodating two or more O-rings. Still alternatively, the O-rings may be over-molded to the plunger using an elastomer, thermoplastic elastomer (TPE) or thermosetting rubber such as Santoprene manufactured by Sonoco or the equivalent.

FIG. 11 is a semi-schematic cross-sectional side view of a fluid handling component 14 mounted to a force generating component 82. In one exemplary embodiment, the force generating component 82 is a prior art component described in anyone of U.S. Pat. Nos. 5,499,972; 5,569,189; and 5,704,911. The fluid handling component 14 provided in accordance with aspects of the present invention is usable by first filling the interior cavity 38 of the housing with a desired dosage. A vial coupler as described in the '911 patent may be used to couple a vial with the fluid handling component 14 to transfer fluid from the vial to the fluid handling component. The connection end 26 of the fluid handling component 14 is then threaded to the receptacle 84 of the force generating component 82. Once mounted, a gap should exists between the drum 86 of the piston 18 and the contact end 74 of the plunger 30. The injector 100 is now ready for use by removing the safety ring (not shown) on the force generating end 82, placing the discharge end 28 against a subject, and squeezing the trigger 88.

The fluid handling component 14 should be discarded after a single use to prevent cross-contaminating a fluid to be injected with an earlier injected fluid, or from contamination in general. This may be accomplished by unthreading the fluid handling component 14 from the force generating component 82 and discarding the same. A second or new injection may be performed by resetting the spring 90 using a reset device as described in U.S. Pat. No. 5,499,972. A new ampule is then filled with a desired dosage, threaded to the force generating component 82, and then injected in the manner previously described.

The fluid handling component 14 may also be used in combination with a disposable force generating component 92, which is shown in FIG. 12 as injector 102. In one exemplary embodiment, the multi-orifice housing 16 is engaged to a disposable force generating component 92 described in U.S. Pat. No. 6,558,348. When use with a disposable force generating component, a modified shaft/plunger 94 as described in the '348 patent is used rather than the relatively shorter plunger 30 described with reference to FIGS. 9 and 10. In one exemplary embodiment, a combination O-ring 80 and push end 76 as described and shown in FIG. 10 may be used with the shaft/plunger 94 shown in FIG. 12.

Although limited embodiments of the needleless injectors and their components have been specifically described and illustrated herein, many modifications and variations will be apparent to those skilled in the art. For example, the various sizes and dimensions may vary for larger or different dosage applications, the material selected could be opaque or semi-opaque, different colors may be used, different radii may be incorporated, etc. Furthermore, a gas driven force generating component may be used instead of the spring driven type. An exemplary gas driven injector is described in U.S. Pat. No. 4,680,027, its contents are expressly incorporated herein by reference. The jet injector assemblies described herein may be sold as separate components, as a combination, and may be pre-filled and pre-packaged with liquid medicaments. Accordingly, it is to be understood that the injectors and their components constructed according to the principles of this invention may be embodied other than as specifically described herein. The invention is also defined in the following claims.

What is claimed is:

1. An ampule for use with a force generating component to discharge fluid subcutaneously comprising a discharge end, a connection end, and a housing body defining an interior cavity disposed therebetween, a plunger slidably movable within the interior cavity of the housing body, and wherein the discharge end comprises a plurality of nozzles passing through at least one raised surface.

2. The ampule as recited in claim 1, wherein the at least one raised surface comprises a continuous ring.

3. The ampule as recited in claim 1, wherein the connection end comprises a plurality of threads.

4. The ampule as recited in claim 1, wherein the discharge end, the connection end, and the housing body are integrally formed as a single structure.

5. The ampule as recited in claim 1, wherein the housing body is made from a thermoplastic material.

6. The ampule as recited in claim 1, wherein the plurality of nozzles comprise two nozzles.

7. The ampule as recited in claim 1; wherein the plurality of nozzles are positioned at an angle to one another.

8. The ampule as recited in claim 1, wherein the connection end is connected to a force generating component.

9. The ampule as recited in claim 8, wherein the force generating component comprises a piston, a spring, and a trigger.

10. The ampule as recited in claim 8, wherein the plunger comprises part of a shaft and the shaft extends through a piston and an injector housing.

11. An ampule for use with a force generating component to discharge fluid subcutaneously comprising a discharge end, a connection end, and a housing body defining an interior cavity disposed therebetween, and wherein the discharge end comprises two or more nozzles positioned at an angle to one another.

12. The ampule as recited in claim 11, further comprising at least one raised surface.

13. The ampule as recited in claim 12, wherein the two or more nozzles pass through the at least one raised surface.

14. The ampule as recited in claim 11, wherein the two or more nozzles terminate as two or more orifices.

15. The ampule as recited in claim 11, wherein fluid flows through the two or more nozzles converge.

16. The ampule as recited in claim 11, further comprising a plunger disposed in the interior cavity of the housing body.

17. The ampule as recited in claim 11, wherein the connection end is connected to a force generating component.

18. The ampule as recited in claim 17, wherein force generating component comprises a piston, a spring, and a trigger.

19. An ampule for use with a force generating component to discharge fluid subcutaneously comprising a discharge end comprising multiple discharge means for discharging fluid, a connection end, a housing body means defining an interior cavity disposed therebetween, and a plunger means for moving fluid through the multiple discharge means to simultaneously discharge the fluid through the multiple discharge means.

20. The ampule as recited in claim 19, wherein the multiple discharge means comprise multiple nozzles and orifices.

21. The ampule as recited in claim 19, wherein the connection end is connected to a force generating component.
